(19) United States
(12) Patent Application Publication
(10) Pub. No.: US 2026/0331328 A1
(43) Pub. Date: Oct. 22, 2026

(54) CONDUCTIVE FILM, MANUFACTURING METHOD FOR CONDUCTIVE FILM, AND ELECTRODE

(71) Applicant: Murata Manufacturing Co., Ltd., Nagaokakyo-shi (JP)

(72) Inventor: Kosuke SUGIURA, Nagaokakyo-shi (JP)

(21) Appl. No.: 19/699,414

(22) Filed: Jun. 5, 2026

Related U.S. Application Data
(63) Continuation of application No. PCT/JP2024/043611, filed on Dec. 10, 2024.

(30) Foreign Application Priority Data
Dec. 11, 2023 (JP) 2023-208781

(51) Int. Cl. H01B 1/12; A61B 5/265; A61B 5/268; H01B 13/32

(52) U.S. Cl. CPC H01B 1/124; A61B 5/265; A61B 5/268; H01B 13/322

(57) ABSTRACT

A conductive film comprising: layered material particles comprising a plurality of layers, and a conductive polymer. The plurality of layers comprising a layer body represented by: MₘXₙ, wherein M is one or more metals selected from Group 3, 4, 5, 6, or 7 and includes Ti; X is a carbon atom, a nitrogen atom, or a combination thereof, n is not less than 1 and not more than 4; and m is greater than n but not more than 5, and a modification or termination T exists on a surface of the layer body, wherein T is at least one selected from the group consisting of a hydroxyl group, a fluorine atom, a chlorine atom, an oxygen atom, and a hydrogen atom.

### CROSS REFERENCE TO RELATED APPLICATIONS

The present application is a continuation of International application No. PCT/JP2024/043611, filed Dec. 10, 2024, which claims priority to Japanese Patent Application No. 2023-208781, filed Dec. 11, 2023, the entire contents of each of which are incorporated herein by reference.

### TECHNICAL FIELD

The present disclosure relates to a conductive film, a manufacturing method for a conductive film, and an electrode.

### BACKGROUND ART

In recent years, MXene has been attracting attention as a new material having conductivity. MXene is a type of so-called two-dimensional materials, and more specifically as will be described later, is a two-dimensional material (layered material) in the form of one or more layers. In general, MXene is in the form of a particle (which can include powder, flake, nanosheet, and the like) of such a two-dimensional material (layered material). Patent Document 1 discloses a bioelectrode formed of a contact material containing MXene.
Patent Document 1: WO 2019/055784 A

### SUMMARY OF THE DISCLOSURE

In Patent Document 1, a composite film of MXene and a conductive polymer is used as an electrode. However, Patent Document 1 does not particularly consider an electrode having a further reduced impedance. An object of the present disclosure is to provide a conductive film having a further reduced impedance, a manufacturing method for the conductive film, and an electrode comprising the conductive film.

According to one aspect of the present disclosure, there is provided a conductive film comprising:

layered material particles comprising a plurality of layers, and a conductive polymer.

The plurality of layers comprising a layer body represented by the following formula:

MₘXₙ
wherein M is one or more metals selected from Group 3, 4, 5, 6, or 7 and includes Ti;
X is a carbon atom, a nitrogen atom, or a combination thereof,
n is not less than 1 and not more than 4; and
m is greater than n but not more than 5, and
a modification or termination T (wherein T is at least one selected from the group consisting of a hydroxyl group, a fluorine atom, a chlorine atom, an oxygen atom, and a hydrogen atom) is present on surface of the layer body.

According to the present disclosure, it is possible to provide a conductive film having a further reduced impedance, a manufacturing method for the conductive film, and an electrode comprising the conductive film.

### BRIEF DESCRIPTION OF THE DRAWINGS

FIG. 1(a) and FIG. 1(b) are schematic sectional views showing layered material particles that can be used in one embodiment of the present disclosure, in which FIG. 1 (a) shows a single-layer MXene particle, and FIG. 1 (b) shows a multilayer (exemplarily, two-layer) MXene particle.

FIG. 2 is a view showing one exemplary mode of layered material particles and a conductive polymer in a conductive film according to the present disclosure.

FIG. 3 is a schematic sectional view showing one embodiment of the electrode of the present disclosure.

FIG. 4 is a schematic sectional view showing another embodiment of the electrode of the present disclosure.

FIG. 5 is a schematic sectional view showing another embodiment of the electrode of the present disclosure.

FIG. 6 is a schematic sectional view showing another embodiment of the electrode of the present disclosure.

FIG. 7 is an SEM observation photograph of a cross section of a composite film of Example 1.

FIG. 8 is an EDX measurement result (Ti) of a cross section of a composite film of Example 1.

FIG. 9 is an EDX measurement result (C) of a cross section of a composite film of Example 1.

FIG. 10 is an EDX measurement result (S) of a cross section of a composite film of Example 1.

FIG. 11 is an SEM observation photograph of a cross section of a composite film of single-layer MXene and PEDOT:PSS.

### DETAILED DESCRIPTION

### Embodiment 1: Conductive Film

Hereinafter, a conductive film in one embodiment of the present disclosure will be described in detail, but the present disclosure is not limited to such an embodiment.

A conductive film in the present embodiment is a conductive film comprising: layered material particles comprising a plurality of layers, and a conductive polymer.

The layers comprising a layer body represented by the following formula:

MₘXₙ
wherein M is one or more metals selected from Group 3, 4, 5, 6, or 7 and includes Ti;
X is a carbon atom, a nitrogen atom, or a combination thereof;
n is not less than 1 and not more than 4; and
m is greater than n but not more than 5, and
a modification or termination T existing on a surface of the layer body, wherein T is at least one selected from the group consisting of a hydroxyl group, a fluorine atom, a chlorine atom, an oxygen atom, and a hydrogen atom.

The layered material (MXene) constituting the layered material particle (MXene particle) may be understood as a layered compound, and is also represented by “MₘXₙTₛ” wherein s is any number, and x or z may be conventionally used instead of s. Typically, n can be 1, 2, 3, or 4, but is not limited thereto.

In the above formula of MXene, M may be only Ti, or may include Ti, and may further include one or more metals of Group 3, 4, 5, 6, or 7 other than Ti. For example, M may include Ti, and may further include at least one selected from the group consisting of Zr, Hf, V, Nb, Ta, Cr, Mo, and Mn. When M includes an element other than Ti, the element other than Ti is more preferably at least one selected from the group consisting of V, Cr, and Mo.

As MXene, those having the above formula MₘXₙ expressed as follows are known.

Ti₂C, Ti₂N, (Ti,V)₂C, (Ti,Nb)₂C, Ti₃C₂, Ti₃N₂, Ti₃(CN), (Ti,V)₃C₂, (Ti₂Nb)C₂, (Ti₂Ta)C₂, (Ti₂Mn)C₂, (V₂Ti)C₂, (Cr₂Ti)C₂, (Mo₂Ti)C₂, (W₂Ti)C₂, Ti₄N₃, (Ti,Nb)₄C₃, (Ti₂Nb₂)C₃, (Ti₂Ta₂)C₃, (V₂Ti₂)C₃, (Cr₂Ti₂)C₃, (Mo₂Ti₂)C₃, (W₂Ti₂)C₃

Typically in the above formula, M can be titanium, or titanium and vanadium, and X can be a carbon atom or a nitrogen atom. For example, the MAX phase which is a precursor of MXene is Ti₃AlC₂ and MXene is Ti₃C₂Tₛ (in other words, M is Ti, X is C, n is 2, and m is 3).

In the present disclosure, MXene may include remaining A atoms at a relatively small amount, for example, at 10 mass % or less with respect to the original A atoms. The remaining amount of A atoms can be preferably 8 mass % or less, and more preferably 6 mass % or less. However, even if the remaining amount of A atoms exceeds 10 mass %, there may be no problem depending on the use and conditions of use of the adsorbent.

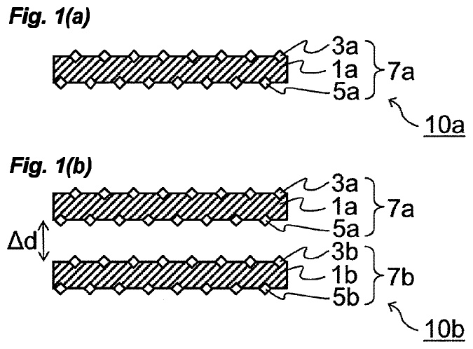

Hereinafter, the MXene particle corresponding to the skeleton of the conductive film according to the present embodiment will be described with reference to FIG. 1.

The MXene particle constituting the conductive film of the present embodiment is an aggregate including a plurality of layers of MXene (multilayer MXene) schematically exemplified in FIG. 1(b). As schematically illustrated in FIG. 1(b), examples of the MXene particle include, but are not limited to, a MXene 10b of two layers. More specifically, each MXene in FIG. 1(b) is a MXene layer 7a having a layer body (MₘXₙ layer) la represented by MₘXₙ and a modification or a termination T 3a, 5a present on a surface of the layer body 1a (more specifically, at least one of both surfaces, facing each other, of each layer). Therefore, the MXene layer (also referred to as “MXene flakes”) 7a is also represented by “MₘXₙTₛ” wherein s is any number.

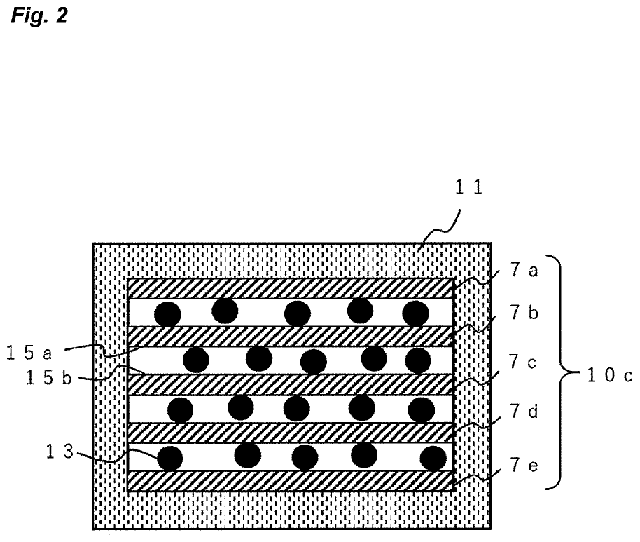

In a MXene 10a shown in FIG. 1(a), the multilayer MXene 10b is individually separated and exists in one layer. In the present embodiment, it is preferable that the multilayer MXene 10b is present as it is rather than the multilayer MXene 10b is individually separated to be present as the MXene 10a shown in FIG. 1(a). FIG. 2 is a schematic sectional view showing a form in which the outermost surface of a multilayer MXene (layered material particle including a plurality of layers) 10c is covered with a conductive polymer 11. FIG. 2 shows a form in which the entire outermost surface of the multilayer MXene 10c is covered with the conductive polymer 11, but the form is not limited to this, and at least a part of the outermost surface of the multilayer MXene may be covered with the conductive polymer. Preferably 50 vol % or more, more preferably 80 vol % or more of the outermost surface of the multilayer MXene, and further preferably all of the outermost surface of the multilayer MXene is covered with the conductive polymer.

FIG. 1(b) illustrates an example in which the MXene layer has two layers, but FIG. 2 illustrates an example in which the MXene layer has five layers, and in FIG. 2, modification or termination T 3a, 5a in FIG. 1(b) are not illustrated. FIG. 2 illustrates a form in which a metal atom (metal cation) 13 is present between layers of MXene, but the metal atom (metal cation) 13 may not be present. As illustrated in FIG. 2, the presence of the conductive polymer 11 around the multilayer MXene 10c makes it possible to ensure the strength and the like of the conductive film while developing low impedance by MXene. The reason why the AC impedance of MXene is low is that there is a large electric double-layer capacitance on the surface of MXene (referring to the surfaces of 7a, 7b in FIG. 1, and also including the mutually opposing surfaces of 7a, 7b, i.e., the surfaces between MXene flakes). It is considered that by using the multilayer MXene as the MXene, when mixed with the conductive polymer, at least the surface (e.g., the MXene surfaces 15a, 15b between the MXene layers 7b, 7c in FIG. 2, and the like) between the MXene flakes in the multilayer MXene is not covered with the conductive polymer, and low impedance can be secured as compared with the case where the entire surface of the single-layer MXene is covered with the conductive polymer.

In the multilayer MXene, two adjacent MXene layers (e.g., 7a and 7b) may not necessarily be completely separated from each other, but may be partially in contact with each other. In addition, the conductive film of the present embodiment is predominantly composed of the multilayer MXene among a total of the MXene particles present, and for example, the ratio of the multilayer MXene in MXene may be, for example, 80 vol % or more, further 90 vol % or more, and still further 95 vol % or more, but it is allowed that a part of the multilayer MXene 10b is separated and the single-layer MXene 10a is slightly present.

Although the present embodiment is not limited, the thickness of each layer (corresponding to the MXene layers 7a, 7b) of MXene is, for example, not less than 0.8 nm and not more than 5 nm, and particularly not less than 0.8 nm and not more than 3 nm (It may mainly vary depending on the number of M atomic layers included in each layer.). For individual laminates of the multilayer MXene that may be included, the interlayer distance (or gap dimension, denoted as Δd in FIG. 1(b)) may be, for example, not less than 0.8 nm and not more than 10 nm, particularly not less than 0.8 nm and not more than 5 nm, and more particularly about 1 nm, and the total number of layers may be not less than 2 and not more than 20,000.

### (Conductive Polymer)

The conductive film of the present embodiment is a composite film including the above-described multilayer MXene and a conductive polymer. MXene exhibits a lower AC impedance than conventional materials. In the case of an electrode including only MXene, although it can be a self-standing film, the strength is low, and it is necessary to improve the strength of the MXene-containing film itself and the adhesion strength to a base material or the like for applying the electrode to the application such as a biosensing electrode, and for this reason, a conductive polymer is contained. By including the conductive polymer, it is possible to reduce the impedance while maintaining the adhesion strength. In particular, in the multilayer MXene in the conductive film of the present embodiment, since the MXene flakes are firmly bonded to each other as compared with the single-layer MXene, the molecules of the conductive polymer are not inserted between the layers. As described above, the reason why the AC impedance of MXene is low is that there is a large electric double-layer capacitance on the surface of MXene (referring to the surfaces of 7a, 7b in FIG. 1, and also including the mutually opposing surfaces of 7a, 7b, i.e., the surfaces between MXene flakes). Therefore, the polymer is preferably disposed so as not to cover the surface of MXene as much as possible. By using the multilayer MXene as the MXene, as described above, the surface between the MXene flakes in the multilayer MXene is not covered with at least the conductive polymer. Therefore, even when the outermost entire surface of the multilayer MXene is covered with the conductive polymer, the impedance is lower than when the entire surface of the single-layer MXene is covered with the conductive polymer.

The polymer contained in the conductive film of the present embodiment may be any type of conductive polymer. Examples of the conductive polymer include polythiophene, poly(3,4-ethylenedioxythiophene), polystyrene sulfonate, polyaniline, and polypyrrole, and one or more selected from the group consisting of these can be used. It is preferable to use PEDOT:PSS (poly(3,4-ethylenedioxythiophene)-polystyrene sulfonate) from the viewpoint of suppressing the resistance of the entire electrode of the conductive film of the present embodiment, particularly the DC resistance to a low level.

The conductive polymer preferably contains a C═O bond (carbon-oxygen double bond). When the conductive polymer has a C═O bond, impedance can be further reduced while adhesion strength is maintained. Examples of the conductive polymer containing a C═O bond include a doped conductive polymer in which a dopant having a C═O bond such as ortho-phthalic acid is doped in the conductive polymer listed above. The presence or absence of the C═O bond can be confirmed by measurement by FT-IR.

The volume ratio of the conductive polymer to the total of MXene and the conductive polymer is preferably not less than 20 vol % and not more than 80 vol %.

### (Metal Atom)

The layered material particle of the present embodiment is preferably a metal atom-containing layered material particle containing a metal atom. By containing a metal atom, the electric double layer capacitance is further increased, and the impedance can be further lowered. When the cation of the metal atom is divalent, it is difficult to sufficiently reduce the impedance, and the metal atom is preferably an alkali metal atom whose cation is monovalent. The metal atom is preferably one or more of lithium, sodium, and potassium. Due to intercalation of the metal atom, the metal atom is preferably present between layers of MXene. The metal atom is preferably present as a metal cation between layers of MXene, preferably between layers of the layered material particle (multilayer MXene) including a plurality of layers. That is, the monovalent metal cation is preferably present between the layers of MXene, preferably between the layers of the multilayer MXene.

It is preferable that the metal atom is contained in an amount of preferably 6.0×10⁻⁴ mol or more per 1 g of the metal atom-containing layered material particle, because the impedance can be further reduced while the adhesion strength is maintained.

### Embodiment 3: Manufacturing Method for Conductive Film

A manufacturing method for a conductive film of the present embodiment comprises:
(a) preparing a precursor represented by the following formula:

MₘAXₙ
(wherein M is one or more metals selected from Group 3, 4, 5, 6, or 7 and includes Ti;
X is a carbon atom, a nitrogen atom, or a combination thereof,
A is one or more elements selected from Group 12, 13, 14, 15, or 16;
n is not less than 1 and not more than 4; and
m is greater than n but not more than 5);
(b) performing an etching treatment to remove at least a part of A atoms from the precursor by using an etching solution containing F⁻;
(c) water washing an etching treatment product obtained by the etching treatment to obtain layered material particles without performing delamination; and
(d) supplying a mixture of the layered material particle and a conductive polymer onto a base material to form a conductive film. By this manufacturing method, a conductive film having a low impedance can be manufactured.

Hereinafter, each step of the manufacturing method will be described in detail.

### Step (a)

First, a predetermined precursor is prepared. The predetermined precursor that can be used in the present embodiment is a MAX phase that is a precursor of MXene, and is represented by the following formula:

MₘAXₙ
(wherein M is one or more metals selected from Group 3, 4, 5, 6, or 7 and includes Ti;
X is a carbon atom, a nitrogen atom, or a combination thereof,
A is one or more elements selected from Group 12, 13, 14, 15, or 16;
n is not less than 1 and not more than 4; and
m is greater than n but not more than 5).

M, X, n, and m are as described in MXene. A is one or more elements selected from Group 12, 13, 14, 15, or 16, and is usually an element of Group A, typically Group IIIA and Group IVA, and more particularly may contain at least one selected from the group consisting of Al, Ga, In, Tl, Si, Ge, Sn, Pb, P, As, S, and Cd, and is preferably Al.

The MAX phase has a crystal structure in which a layer formed by A atoms is located between two layers represented by MₘXₙ (which may have such a crystal lattice that each X is located in an octahedral array of M). When typically m=n+1, but not limited thereto, the MAX phase includes repeating units in which each one layer of X atoms is disposed in between adjacent layers of n+1 layers of M atoms (these are also collectively referred to as an “MₘXₙ layer”), and a layer of A atoms (“A atom layer”) is disposed as a layer next to the (n+1)th layer of M atoms.

The MAX phase can be produced by a known method. For example, a TiC powder, a Ti powder, and an Al powder are mixed with a ball mill, and the resulting mixed powder is fired in an Ar atmosphere to obtain a fired body (block-like MAX phase). Thereafter, the fired body obtained is crushed with an end mill to obtain a powdery MAX phase for the next step.

### Step (b)

An etching treatment for removing at least a part of A atoms from the precursor using an etching solution containing F⁻ is performed.

By selectively etching (removal and layer separation in some cases) A atoms (and part of M atoms) from the MAX phase, the A atom layer (and part of M atoms in some cases) is removed, and the surface of the exposed MₘXₙ layer is modified by hydroxyl groups, fluorine atoms, chlorine atoms, oxygen atoms, hydrogen atoms, etc., existing in an etching liquid (usually, an aqueous solution of a fluorine-containing acid is used, but not limited thereto), so that the surface is terminated.

The etching is carried out using an etching liquid containing F⁻, and a method using, for example, a mixed liquid of lithium fluoride and hydrochloric acid, a method using hydrofluoric acid, or the like may be used.

### Step (c)

The etching treatment product (an intercalation treatment product when an intercalation treatment including mixing a compound containing a metal atom and an etching treatment product is performed after the etching treatment (b) but before the water washing (c)) obtained by the etching treatment is washed with water to obtain layered material particles without performing delamination. Specifically, after etching (or intercalation treatment), decantation is performed using pure water and a centrifuge to clean the remaining acid. In the present embodiment, delamination is not performed so as to obtain a multilayer MXene rather than a single-layer/few-layer MXene. After decantation, the precipitate collected by centrifugation can be obtained as a clay-like multilayer MXene, or the clay-like MXene can be dried by freeze-drying to obtain a powdery multilayer MXene.

(Intercalation treatment)

In the case of obtaining a metal atom-containing layered material particle, a metal atom intercalation treatment including mixing a compound containing a metal atom with a precursor is performed at the time of the etching treatment in the above (b), or a metal atom intercalation treatment including mixing a compound containing a metal atom with an etching treatment product is performed after the etching treatment in the above (b) but before the water washing in the above (c).

When the metal atom intercalation treatment is performed at the time of the etching treatment, examples of the metal compound containing a monovalent metal ion include those used in the following intercalation treatment. The content of the metal compound containing a monovalent metal ion in the etching solution is preferably 0.001 mass % or more. The content is more preferably 0.01 mass % or more, and further preferably 0.1 mass % or more. On the other hand, from the viewpoint of dispersibility in the solution, the content of the metal compound containing a monovalent metal ion in the etching solution is preferably 10 mass % or less, and more preferably 1 mass % or less.

When the metal atom intercalation treatment is performed after the etching treatment (b) but before the water washing (c), for example, the metal atom intercalation treatment may be performed as follows.

For example, an intercalation treatment of monovalent metal ion including a step of mixing the etching treatment product obtained by the etching treatment with a metal compound containing a monovalent metal ion is performed. In performing the intercalation treatment, the powder obtained through the above-described washing step and redispersed in pure water once may be used instead. Examples of the monovalent metal ion constituting the metal compound containing a monovalent metal ion include alkali metal ions such as a lithium ion, a sodium ion, and a potassium ion, a copper ion, a silver ion, and a gold ion. Examples of the metal compound containing a monovalent metal ion include an ionic compound in which the metal ion and a cation are bonded. Examples include an iodide, a phosphate, a sulfur-containing salt such as a sulfate, a nitrate, an acetate, and a carboxylate of the metal ion. The monovalent metal ion is preferably a sodium ion, and the metal compound containing a monovalent metal ion is preferably a metal compound containing a sodium ion, more preferably an ionic compound of a sodium ion, and further preferably one or more of an iodide, a phosphate, and a sulfide salt of a sodium ion. When a sodium ion is used as the metal ion, it is considered that an ideal multilayer MXene in which sodium is inserted between layers but is not formed into a single layer can be obtained.

The content of the compound containing a metal atom in the formulation for intercalation treatment is preferably 0.001 mass % or more. The content is more preferably 0.01 mass % or more, and further preferably 0.1 mass % or more. On the other hand, from the viewpoint of dispersibility in the solution, the content of the compound containing a metal atom is preferably 10 mass % or less, and more preferably 1 mass % or less.

A specific method of the intercalation treatment is not particularly limited, and for example, the compound containing a metal atom may be mixed with the water medium clay of MXene and stirred, or may be allowed to stand. For example, stirring at room temperature can be mentioned. Examples of the stirring method include a method using a stirring bar of a stirrer or the like, a method using a stirring blade, a method using a mixer, a method using a centrifuge, and the like, and the stirring time can be set according to the production scale of the adsorbent, and for example, the stirring time is set between 12 hours and 24 hours.

### Step (d)

A mixture of the layered material particle and the conductive polymer is supplied onto a base material to form a conductive film.

A mixture of the layered material particle and the conductive polymer can be obtained, for example, as exemplified below. For example, a MXene aqueous dispersion or a MXene dispersion dispersed in an organic medium liquid, in which the layered material particle (MXene particle) is present in a medium liquid, or a MXene powder is mixed with the above-described conductive polymer to obtain a mixture. The medium liquid of the MXene aqueous dispersion is typically water, and in some cases, other liquid substances may be contained in a relatively small amount (e.g., 30 mass % or less, preferably 20 mass % or less based on the whole mass) in addition to water. Examples of the organic medium liquid include N-methylpyrrolidone, N-methylformamide, N,N-dimethylformamide, ethanol, methanol, dimethylsulfoxide, ethylene glycol, and acetic acid.

The layered material particle and the conductive polymer can be stirred using a dispersing device such as a homogenizer, a propeller stirrer, a thin film swirling stirrer, a planetary mixer, a mechanical shaker, or a vortex mixer.

Applying a slurry, which is a mixture of the layered material particle and the conductive polymer, to at least a part of a surface of the base material (when there is a silver/silver chloride electrode part substrate, for example, the surface of the silver/silver chloride electrode part formed on the surface of the base material) can be mentioned. The applying method is not limited, and examples thereof include a method of performing spray application using a nozzle such as a one-fluid nozzle, a two-fluid nozzle, or an air brush, a method such as slit coating using a table coater, a comma coater, or a bar coater, screen printing, or metal mask printing, and an applying method by spin coating, immersion, or dropwise application.

The formation and drying of the conductive film can be appropriately repeated until a desired conductive film thickness is obtained. For example, the combination of the spraying and the drying may be repeated multiple times. In the conductive film, a liquid component coming from the liquid medium of the slurry may remain, and may substantially not remain.

Drying may be performed under mild conditions such as natural drying (typically, placing in an air atmosphere at normal temperature and normal pressure) or air drying (blowing air), or may be performed under relatively active conditions such as hot air drying (blowing heated air), thermal drying, and/or vacuum drying. In the present embodiment, “drying” means removing the medium liquid that may be present in the precursor. The drying may be performed at a temperature of 400 °C or less using, for example, a normal pressure oven or a vacuum oven. Drying may be performed, for example, at not lower than 30 °C and not higher than 200 °C for not less than 30 minutes and not more than 24 hours.

### Embodiment 2: Electrode

The electrode according to the present embodiment includes the conductive film. The electrode of the present embodiment only needs to include the conductive film, and is not limited to a specific form. The electrode may be formed of only the conductive film, or may include the conductive film and, for example, a base material. Examples of the electrode include an electrode in a solid state and an electrode in a flexible and soft state.

In the electrode of the present embodiment, the conductive film may be exposed to the external environment so as to be in direct contact with the object to be measured, or a gel capable of permeating ions, a film including a porous membrane, or the like may be formed as another laminated layer. The porous membrane may be a membrane having a large number of fine pores and selectively transmitting ions and molecules having a size smaller than the pore diameter. The material of such another laminated layer is not particularly limited, and may be formed of an organic material, an inorganic material, or a mixture thereof. Examples of the organic material include polymers such as hydrophilic polymers, and examples of the inorganic material include ceramics, or a combination thereof. The film thickness of such another laminated layer can be, for example, not less than 0.1 μm and not more than 300 μm. The porous membrane may have, for example, an average pore diameter of not less than 1 nm and not more than 1 μm. Examples of the porous membrane cited for different pore shapes include an aggregated particulate porous membrane, a network porous membrane, a fibrous porous membrane, a porous membrane having a plurality of isolated and/or tubular pores, and a porous membrane having a honeycomb structure.

When the electrode of the present embodiment has a base material, the conductive film and the base material may be in direct contact with each other. The material of the base material is not particularly limited. The base material may be formed of a conductive material. Examples of the conductive material include at least one material of a metal material such as gold, silver, copper, platinum, nickel, titanium, tin, iron, zinc, magnesium, aluminum, tungsten, or molybdenum, or a conductive polymer. The base material may have a conductive layer, such as a metal film, different from the conductive film according to the present embodiment, on a contact surface with the conductive film according to the present embodiment. Alternatively, the base material may be formed of an organic material. Examples of the organic material include flexible organic materials, and examples thereof include thermoplastic polyurethane elastomer (TPU), a PET film, and a polyimide film.

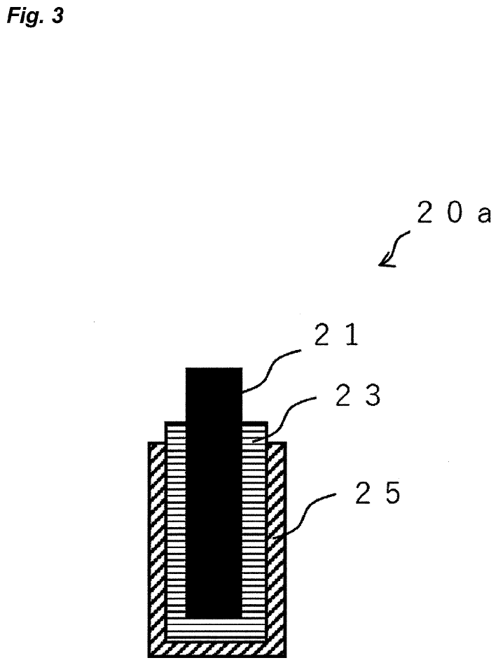

As one preferable mode, the electrode according to the present embodiment includes a base material, a silver/silver chloride electrode part, and the conductive film (composite film). One mode of the electrode according to the present embodiment is an electrode in which a silver/silver chloride electrode part is disposed on at least a part of a surface of the base material, and a conductive film is disposed on at least a part of the surface of the silver/silver chloride electrode part exposed to the external environment. For example, as shown in a schematic sectional view in FIG. 3, an electrode 20a in which a silver/silver chloride electrode part 23 is disposed on the surface of an electrode base material 21, and a conductive film 25 is disposed on the surface of the silver/silver chloride electrode part 23 is exemplified. As shown in FIG. 3, an electrode is exemplified in which the base material 21 has a tip part and a longitudinal direction, the silver/silver chloride electrode part 23 is disposed on the surfaces of the tip part and a side face part of the base material 21, and the conductive film 25 is disposed on approximately 50% or more of the surface of the silver/silver chloride electrode part 23 exposed to the external environment.

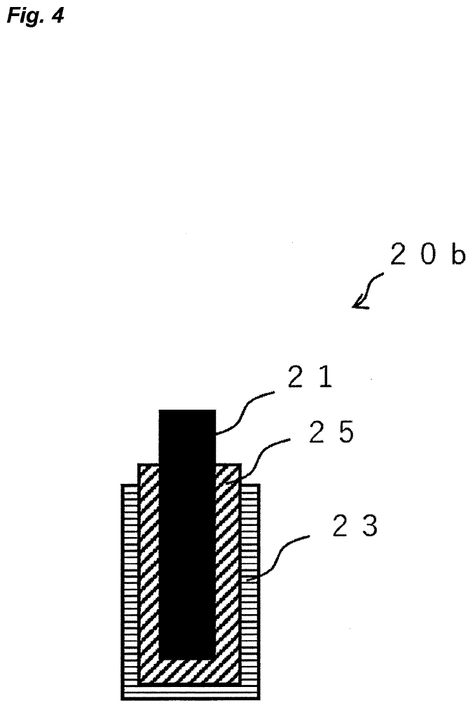

Another mode of the electrode according to the present embodiment is an electrode in which a conductive film is disposed on at least a part of a surface of the base material, and a silver/silver chloride electrode part is disposed on at least a part of a surface of the conductive film exposed to the external environment. As an example thereof, for example, as shown in a schematic sectional view in FIG. 4, an electrode 20b in which a conductive film 25 is disposed on the surface of the electrode base material 21, and the silver/silver chloride electrode part 23 is disposed on the surface of the conductive film 25 is exemplified. As shown in FIG. 4, an electrode is exemplified in which the base material 21 has a tip part and a longitudinal direction, the conductive film 25 is disposed on the surface including the tip part of the base material 21, and the silver/silver chloride electrode part 23 is disposed on about 50% or more of the surface of the conductive film 25 exposed to the external environment.

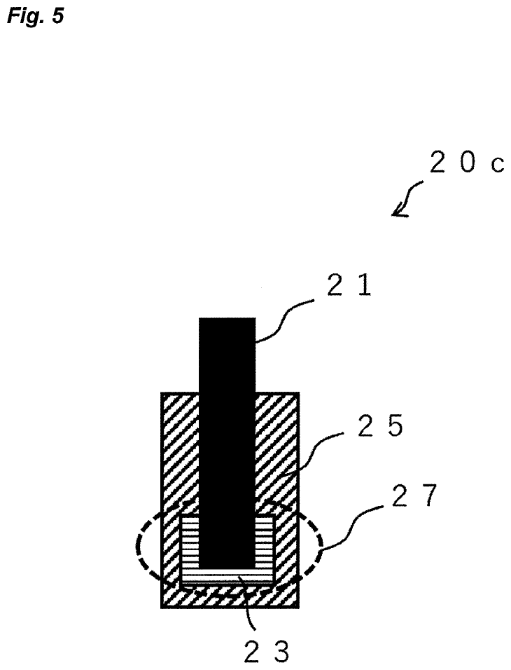

As a modified example of FIG. 3, as shown in the schematic sectional view of FIG. 5, an electrode 20c in which the ratio of the silver/silver chloride electrode part 23 disposed on the surface of the electrode base material 21 is smaller than that in FIG. 3 is exemplified. As shown in FIG. 5, an electrode is exemplified in which the base material 21 has a tip part and a longitudinal direction, the silver/silver chloride electrode part 23 is disposed on the surface including the tip part of the base material 21, and the conductive film (film containing layered material particle) 25 is disposed on the entire surface of the silver/silver chloride electrode part 23 exposed to the external environment. In one mode, the electrode of the present embodiment is, for example, a contact pin in which the base material has a tip part and a longitudinal direction as shown in FIG. 5, and the silver/silver chloride electrode part 23 is formed only in a tip region 27 of the contact pin, and the conductive film 25 is disposed so as to cover the entire surface of the silver/silver chloride electrode part 23 exposed to the external environment. When the electrode of such a mode is used, for example, as a biosensing electrode, the electrode is excellent in biocompatibility because the surface to be in contact with the skin is a conductive film. In addition, as shown in FIG. 5, it is considered that by suppressing the ratio of the silver/silver chloride electrode part in the electrode and covering the silver/silver chloride electrode part with the conductive film, deterioration of the silver/silver chloride electrode part is suppressed, and the life of the electrode can be extended.

In the electrode according to the present embodiment, as shown in FIG. 3 and the like, it is preferable that the silver/silver chloride electrode part and the conductive film are in direct contact at least partially.

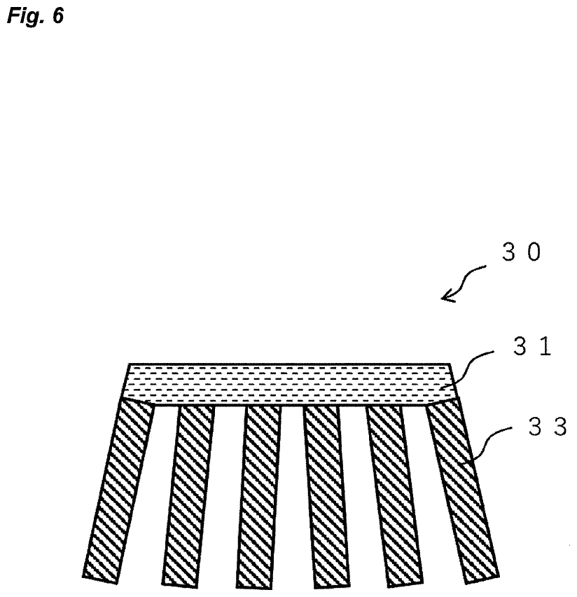

Another mode of the electrode according to the present embodiment is a brush-shaped electrode having a support and a plurality of contact pins to be brought into contact with an object to be measured, in which the silver/silver chloride electrode part is disposed on at least a part of a surface of the base material in a region including the tip of the contact pin, and the conductive film is disposed on at least a part of a surface of the silver/silver chloride electrode part exposed to the external environment. For example, as illustrated in FIG. 6, a brush-shaped electrode 30 in which a plurality of contact pins 33 are connected to a support 31 is exemplified, and each of the plurality of contact pins 33 can take any one of the modes of FIGS. 3 to 5 described above, for example.

FIGS. 3 to 6 are explanatory diagrams for easy understanding, and the shape, the size such as film thickness, the mutual size ratio, the number of contact pins, intervals, arrangements, and the like of the electrode base material, the silver/silver chloride electrode part, and the conductive film in these drawings may be different from those of actual electrodes. The electrode according to the present embodiment has been described with reference to the drawings, but the electrode according to the present embodiment is not limited to the modes illustrated in the drawings. For example, the cross section of the contact pin of the brush-shaped electrode may be not only the rectangular shape illustrated in the above drawings but also a tapered triangular shape or a tapered trapezoidal shape. In addition, the tip of the contact pin may be a flat surface or a convex curved surface.

### (Manufacturing Method for Electrode)

The electrode according to the present embodiment can be manufactured by the method described below, for example, when the electrode has a silver/silver chloride electrode part substrate. However, the manufacturing method for an electrode according to the present embodiment is not limited to this embodiment.

One example of the manufacturing method for an electrode according to the present embodiment includes:
(A) forming a film containing a mixture of a multilayer MXene and a conductive polymer on at least a part of the surface (surface exposed to the external environment) of the silver/silver chloride electrode part formed on the surface of the base material using the mixture or
(B) forming a film containing the mixture on at least a part of the surface of the base material using a mixture of a multilayer MXene and a conductive polymer, and then forming a silver/silver chloride electrode part on at least a part of the surface (surface exposed to an external environment) of the film containing the mixture.

The mixture of a multilayer MXene and a conductive polymer is as described in the manufacturing method for a conductive film. In both the step (A) and the step (B), the silver/silver chloride electrode part can be formed by the same method as a usual method for forming a silver/silver chloride electrode. The mixture of a multilayer MXene and a conductive polymer may be supplied as it is or appropriately adjusted (for example, dilution with a medium liquid, or addition of a binder), to at least a part of the surface of the silver/silver chloride electrode part in the case of the step (A), and may be supplied to at least a part of the surface of the base material in the case of the step (B). As one example of a supplying method, application can be mentioned. The applying method and drying after application are as described in the manufacturing method for a conductive film.

Although the conductive film and the electrode of the present disclosure have been described in detail above, various modifications are possible. It should be noted that the conductive film and the electrode of the present disclosure may be manufactured by a method different from the manufacturing method in the above-described embodiment.

### EXAMPLES

Hereinafter, the present disclosure will be described more specifically with reference to examples. The present disclosure is not limited by the following examples, and can be implemented with appropriate modifications within a range that can be consistent with the above-described and later-described gist, and any of them is included in the technical scope of the present disclosure.

### Example 1

### 1. Preparation of MXene

First, layered material particles (MXene particles) were obtained by sequentially performing (1) preparation of precursor (MAX), (2) etching of precursor, (3) washing after etching, (4) Na intercalation, (5) washing after intercalation treatment, and (6) powderization, which are described in detail below.

(1) Preparation of precursor (MAX)

TiC powder, Ti powder, and Al powder (all manufactured by Kojundo Chemical Laboratory Co., Ltd.) were placed in a ball mill containing zirconia balls at a molar ratio of 2:1:1 and mixed for 24 hours. The obtained mixed powder was fired in an Ar atmosphere at 1350 °C for 2 hours. The fired body (block-like MAX) thus obtained was crushed with an end mill to a maximum size of 40 μm or less. Thereby, Ti₃AlC₂ particles were obtained as a precursor (powdery MAX).

### (2) Etching of Precursor (MAX)

Using the Ti₃AlC₂ particle (powder) prepared by the above method, etching was performed under the following etching conditions to obtain a solid-liquid mixture (slurry) containing a solid component derived from the Ti₃AlC₂ powder.

### (Etching Conditions)

Precursor: Ti₃AlC₂ (sieving with a mesh size of 45 μm)
Etching solution composition: 49% HF 6 mL,
H₂O 18 mL
HCl(12M) 36 mL
Amount of precursor input: 3.0 g
Etching container: 100 mL iboy
Etching temperature: 35 °C
Etching time: 24 h
Stirrer rotation speed: 400 rpm
(3) Washing after etching

The above slurry was equally divided into two portions and inserted into two 50 mL centrifuge tubes. Then, centrifugation was performed at 3500 G for 5 minutes using a centrifuge, and then the supernatant was discarded. Thereafter, (i) 35 mL of pure water was added to the remaining precipitate in each centrifuge tube, (ii) stirring was performed by manually shaking, (iii) centrifugation was performed at 3500 G for 5 minutes, and (iv) the supernatant was removed. The steps (i) to (iv) were repeated 10 times. Finally, centrifugation was performed at 3500 G for 5 minutes to obtain a Ti₃C₂Tₛ-water medium clay. The Ti₃C₂Tₛ-water medium clay was freeze-dried to obtain a powder of MXene after etching and washing.

### (4) Na Intercalation

For intercalation of sodium, 1.6 g of MXene powder after etching and washing and 40 g of pure water were weighed and stirred. Separately from this, 2.16 g of NaCl powder and 40 g of pure water were weighed and stirred. The two solutions were stirred.
Intercalation container: 100 mL iboy
Temperature: not lower than 20 °C and not higher than 25 °C (room temperature)
Time: 12 h
Stirrer rotation speed: 800 rpm
(5) Washing after intercalation treatment

Each of the slurries obtained by the intercalation was transferred to a centrifuge tube, (i) 40 mL of pure water was added, (ii) centrifugation was performed under the condition of 3500 G using a centrifuge, and (iii) a supernatant was separated and removed. The operations (i) to (iii) were repeated five times in total to remove excessive Na, thereby obtaining a Na intercalated MXene clay (sodium intercalated multilayer MXene clay) as an intercalation treatment product.

### (6) Powderization

The intercalation treatment product was appropriately divided into petri dishes and frozen at −18 °C or lower. Thereafter, the intercalation treatment product was dried with a freeze dryer for 24 hours or more to obtain a powder.

### 2. Preparation of Composite Slurry of MXene and Conductive Polymer

The powder obtained in the above (6) (1.346 g) and 3.158 g of Taycatron PSCM1 manufactured by TAYCA Co., Ltd. were weighed in a 105 mL wide-mouth poly container, and stirred for 60 seconds under the conditions of revolution 1340 rpm and rotation 1340 rpm using a rotation and revolution mixer to obtain a composite slurry of a MXene and a conductive polymer. As shown in Table 1 as “Taycatron PSCM1 (50 vol %)”, the volume ratio of the conductive polymer to (MXene+conductive polymer) was 50 vol %.

### 3. Preparation of Composite Electrode of MXene and Conductive Polymer

### [For Impedance Measurement]

A tip of a conductive rubber brush electrode manufactured by Datwyler was dip-coated with the composite slurry obtained in the above “2. Preparation of composite slurry of MXene and conductive polymer” to form a composite film. The brush electrode was previously subjected to plasma cleaning for 1 minute under desired conditions using oxygen plasma. The brush electrode dip-coated with the composite slurry was dried at 80 °C for 2 hours in a normal pressure oven to obtain an electrode for impedance measurement.

### [For Cross-Cut Test]

The composite slurry obtained in the above “2. Preparation of composite slurry of MXene and conductive polymer” was applied to a polyimide film with a film coater to form a composite film. The polyimide film was previously subjected to plasma cleaning for 1 minute under desired conditions using oxygen plasma. After the film formation, drying was performed at 80 °C for 2 hours using a normal pressure oven to obtain an electrode for a cross-cut test.

### Example 2

An electrode was obtained in the same manner as in Example 1 except that Li intercalation was performed under the following conditions instead of the Na intercalation.

### (Conditions of Li Intercalation)

For lithium intercalation, 1.6 g of MXene powder after etching and washing and 40 g of pure water were weighed and stirred. Separately from this, 1.6 g of LiCl powder and 40 g of pure water were weighed and stirred. The two solutions were stirred.
Intercalation container: 100 mL iboy
Temperature: not lower than 20 °C and not higher than 25 °C (room temperature)
Time: 12 h
Stirrer rotation speed: 800 rpm

### Example 3

An electrode was obtained in the same manner as in Example 1 except that the intercalation was not performed after the etching, and washing was conducted by performing decantation with pure water using a centrifuge, and the precipitate obtained by centrifugation was collected as a non-intercalated multilayer MXene.

### Comparative Example 1

The same procedure as in Example 2 was performed up to Li intercalation. An electrode was obtained in the same manner as in Example 2 except that delamination was performed on the Li intercalation treatment product under the following conditions.

### (I) Delamination and Water Washing

The slurry obtained by performing Li intercalation was charged into a 50 mL centrifuge tube, centrifuged under the condition of 3500 G using a centrifuge, and then the supernatant was discarded. Next, (i) 40 mL of pure water was added to the remaining precipitate, and the mixture was stirred for 15 minutes with a shaker, then (ii) centrifuged at 3500 G, and (iii) the supernatant was collected as a solution containing single-layer/few-layer MXene. The operations (i) to (iii) were repeated four times in total to obtain a supernatant containing single-layer/few-layer MXene. Furthermore, the supernatant was centrifuged under the conditions of 4300 G and 2 hours using a centrifuge, and then the supernatant was discarded to obtain MXene clay containing single-layer/few-layer MXene as the remaining precipitate.

### (II) Powderization

The delaminated MXene was appropriately subdivided on petri dishes and frozen at −18 °C or lower. Thereafter, the MXene was dried with a freeze dryer for 24 hours or more to obtain a powder.

### Comparative Example 2

An electrode was obtained in the same manner as in Comparative Example 1 except that the powderization of the above (II) was not performed and a slurry (water slurry) was supplied for formation of an electrode.

The manufacturing conditions for MXene and conductive polymers used in Examples 1 to 3 and Comparative Examples 1 and 2 are shown in Table 1.

TABLE 1

| | MXene | | | | |
|---|---|---|---|---|---|
| | | | | Powderization by | |
| | | Intercalation | Delamination | freeze drying | Conductive polymer |
| Example 1 | Na intercalation | Na | Not performed | Performed | Taycatron PSCM1 (50 vol %) |
| | Multilayer MXene | | | | |
| Example 2 | Li intercalation | Li | Not performed | Performed | Taycatron PSCM1 (50 vol %) |
| | Multilayer MXene | | | | |
| Example 3 | No intercalation | Not performed | Not performed | Performed | Taycatron PSCM1 (50 vol %) |
| | Multilayer MXene | | | | |
| Comparative | Single-layer MXene | Li | Performed | Performed | Taycatron PSCM1 (50 vol %) |
| Example 1 | | | | | |
| Comparative | Single-layer MXene | Li | Performed | Not performed | Taycatron PSCM1 (50 vol %) |
| Example 2 | (water slurry) | | | | |

### 4. Evaluation of Structure of Conductive Film

### [SEM-EDX Observation]

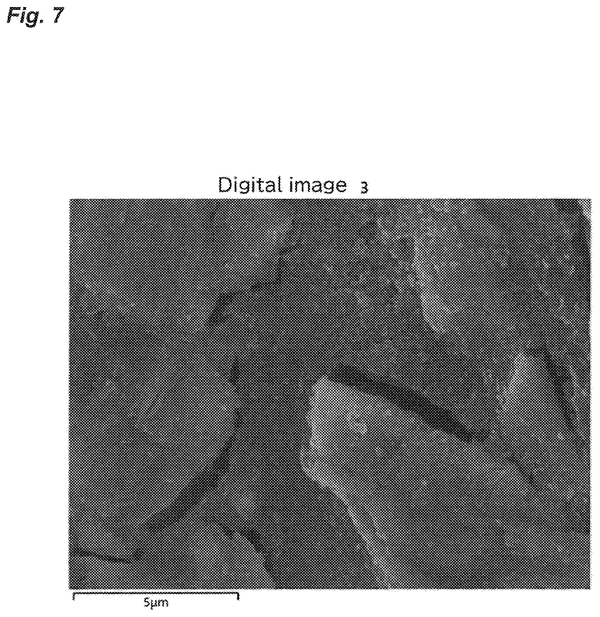

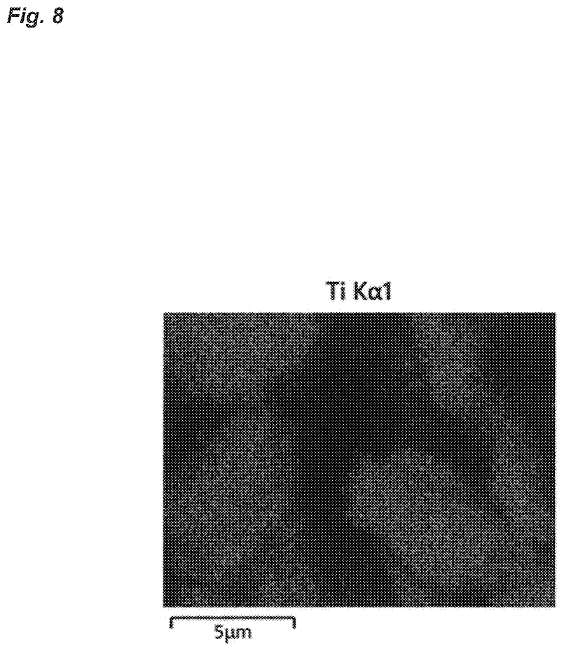

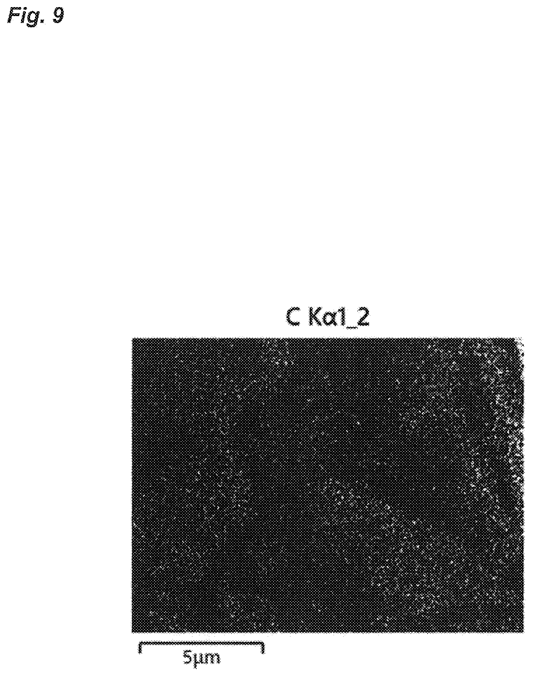

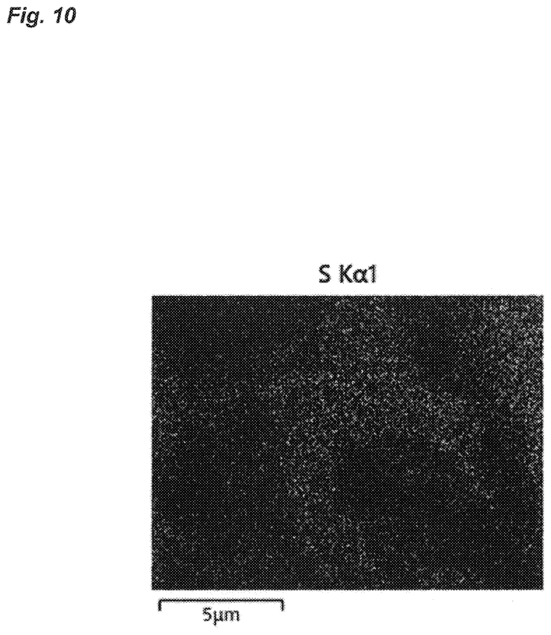

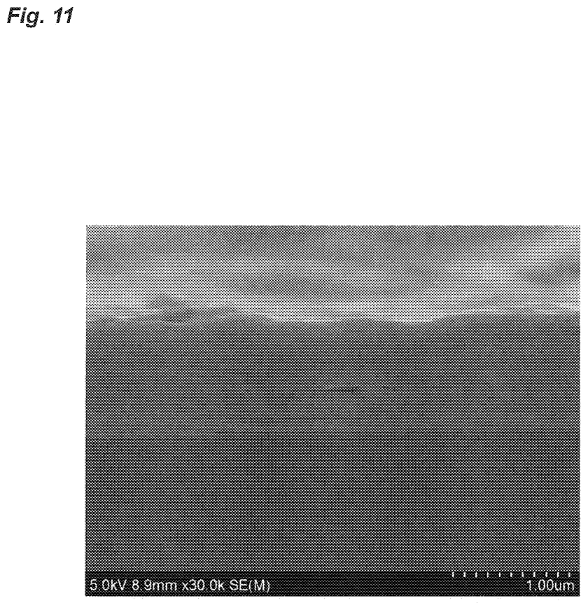

The composite film obtained in Example 1 was polished so that a cross section in a film thickness direction of the composite film could be observed. Then, a cross section of the composite film in the film thickness direction was observed by SEM-EDX. The SEM observation photograph is shown in FIG. 7. In FIG. 7, it is considered that the object having a low contrast and a rock shape is the multilayer MXene, and a high-contrast part around the object is PEDOT:PSS. As evidence, in EDX, as shown in FIG. 8, Ti was frequently detected at the position of the rock shaped object, and thus the shaped object is considered to be MXene. On the other hand, in the part with high contrast in FIG. 7, as illustrated in FIGS. 9 and 10 in which the locations of C and S are indicated by white points, respectively in EDX, a large amount of S is detected, and C is also detected, and thus it is considered that the part is PEDOT:PSS. In addition, it can be seen from FIG. 7 that at least a part of the outermost surface of the multilayer MXene (layered material particle including a plurality of layers) is covered with the conductive polymer. For reference, FIG. 11 shows an SEM observation photograph of a cross section of a composite film of single-layer MXene (layered material particle including one layer) and PEDOT:PSS (the product number is different from that of the material used in this Example, but the basic structure is identical). From the comparison between FIG. 7 and FIG. 11, it can be seen that the case of using the multilayer MXene and the case of using the single-layer MXene as MXene are clearly different even in the cross section in the film thickness direction of the same composite film. It can be seen from FIG. 7 that the conductive film according to the present embodiment is composed of multilayer MXene (the ratio of multilayer MXene is 80 vol % or more).

The composite film obtained in Example 1 was evaluated by X-ray diffraction (XRD), and it was separately confirmed that the interlayer distance was wider than that of the composite film obtained without performing intercalation. From this, it is considered that metal atoms (metal cations) are present between layers in the composite film obtained by preferably performing intercalation.

Further, measurement was performed by FT-IR (Fourier transform infrared spectroscopy) using the conductive film of Example 1, and it was confirmed that the conductive film had a C═O bond (carbon-oxygen double bond).

### 5. Evaluation of Characteristics

Using the electrodes obtained in Examples 1 to 3, Comparative Example 1, and Comparative Example 2, measurement of impedance and evaluation of strength (adhesion strength with a base material) were performed as the evaluation of conductivity as follows.

### [Measurement of Impedance]

On a biosheet (product number: OHMTS01212) manufactured by WetLab, gel electrodes (product number: 019-415200) manufactured by Natus were installed as a counter electrode and a reference electrode. In addition, the impedance measuring electrode produced in the above “3. Preparation of composite electrode of MXene and conductive polymer” was installed as a working electrode.

Impedance measurement was performed using an electrochemical measuring instrument (product number: PGSTAT302N) manufactured by Metrohm. Detailed conditions are shown below. Basically, a signal around 10 Hz is important for brain waves, but a signal of 0.1 Hz may also be used. For example, the signal of slow potential, which is a hint for diagnosis of epilepsy, is 0.1 Hz. Therefore, in the measurement, values of 0.1 Hz and 10 Hz were used as representative values for analysis.

### (Impedance Measurement Conditions)

Frequency domain: 0.1 to 104 Hz
Number of plots: 51 points
Voltage: 10 mVrms

### [Cross-Cut Test]

The cross-cut test was performed by a method in accordance with JIS K 5600-5-6:1999. Specifically, in the composite film of the electrode for the cross-cut test prepared in the above “3. Preparation of composite electrode of MXene and conductive polymer”, a grid pattern cut was made. An operation of attaching and removing a cellophane adhesive tape to and from the part where the above cut was made was conducted. Then, it was confirmed whether the composite film (electrode) was peeled off from the polyimide film.

The measurement results are shown in Table 2.

TABLE 2

| | 0.1 Hz Impedance | 10 Hz Impedance | Cross-cut test |
|---|---|---|---|
| | [Ohm] | [Ohm] | result |
| Example 1 | 513 | 459 | No peeling |
| Example 2 | 525 | 450 | No peeling |
| Example 3 | 978 | 485 | No peeling |
| Comparative | The slurry viscosity was too high to form a film. | | |
| Example 1 | | | |
| Comparative | 20505 | 3307 | Peeled off |
| Example 2 | | | |

The results of Tables 1 and 2 are discussed below. The composite films (electrodes) of Examples 1 to 3 showed a lower AC impedance than the composite films (electrodes) of Comparative Examples 1, 2. This is considered to be because, as described above, in the multilayer MXene, the flakes of MXene are firmly bonded to each other as compared with the single-layer MXene, polymer molecules are not inserted between layers, and the MXene surface between flakes is not covered with the polymer, so that the impedance is lower than when the single-layer MXene is used. Among Examples 1 to 3, Examples 1, 2 containing a metal atom had lower impedance than Example 3. This is considered to be because the electric double-layer capacitance is further increased by containing a metal atom.

On the other hand, the single-layer MXene powder used in Comparative Example 1 had a large specific surface area, the polymer and the MXene flakes interacted with each other, and the sum of the interactions between the respective MXene flakes and the polymer appeared as an increase in viscosity of the slurry. As a result, a film could not be formed, and characteristics could not be evaluated. In Comparative Example 2, the fact that the surface of the MXene flakes was covered with the polymer and it became difficult to function as an electrode, and the fact that the crystallization of PEDOT collapsed due to the addition of water due to the use of the water slurry and the conductivity decreased are considered as two reasons for the increase in impedance.

As in Examples 1 to 3, by making a composite film with a polymer using a multilayer MXene, the adhesion strength to a base material or the like can be improved as compared with the case of using only MXene, and as a result, application, for example, to a biosensing electrode or the like is possible.

This application claims priority based on Japanese Patent Application No. 2023-208781. Japanese Patent Application No. 2023-208781 is incorporated herein by reference.

The conductive film of the present disclosure can be utilized for any suitable application, and can be preferably used as, for example, an electrode and even a biological signal sensing electrode.

REFERENCE SIGNS LIST

| 1a, 1b | Layer body (MmXn layer) |
|---|---|
| 3a, 5a, 3b, 5b | Modification or termination T |
| 7a, 7b, 7c, 7d, 7e | MXene layer |
| 10a, 10b, 10c | MXene particle (layered material particle) |
| 11 | Conductive polymer |
| 13 | Metal atom (metal cation) |
| 15a, 15b | MXene surface between MXene layers |
| 20a, 20b, 20c, 30 | Electrode |
| 21 | Electrode base material |
| 23 | Silver/silver chloride electrode part |
| 25 | Conductive film |
| 27 | Tip region of contact pin |
| 31 | Support |
| 33 | Contact pin |

1. A conductive film comprising:
layered material particles comprising a plurality of layers predominantly, and a conductive polymer,
the plurality of layers comprising a layer body represented by:
MₘXₙ
wherein M is one or more metals selected from Group 3, 4, 5, 6, or 7 and includes Ti;
X is a carbon atom, a nitrogen atom, or a combination thereof;
n is not less than 1 and not more than 4; and
m is greater than n but not more than 5, and
a modification or termination T exists on a surface of the layer body, wherein T is at least one selected from the group consisting of a hydroxyl group, a fluorine atom, a chlorine atom, an oxygen atom, and a hydrogen atom.

2. The conductive film according to claim 1, wherein the conductive polymer is one or more selected from the group consisting of polythiophene, poly(3,4-ethylenedioxythiophene), polystyrene sulfonic acid, polyaniline, and polypyrrole.

3. The conductive film according to claim 1, wherein the conductive polymer includes a C═O bond.

4. The conductive film according to claim 1, wherein the layered material particle is a metal atom-containing layered material particle containing a metal atom.

5. The conductive film according to claim 4, wherein the metal atom is one or more of lithium, sodium, and potassium.

6. The conductive film according to claim 4, wherein the metal atom is present as a metal cation between layers of the layered material particle comprising a plurality of layers.

7. The conductive film according to claim 4, wherein the metal atom is contained in the conductive film in an amount of 6.0×10⁻⁴ mol or more per 1 g of the metal atom-containing layered material particle.

8. The conductive film according to claim 1, wherein a ratio of the layered material particles comprising the plurality of layers predominately in the layered material particles included in the conductive film is 80 vol % or more.

9. The conductive film according to claim 1, wherein at least a part of an outermost surface of the layered material particles is covered with a conductive polymer.

10. An electrode comprising the conductive film according to claim 1.

11. The electrode according to claim 10, further comprising: a base material supporting the layered material particles; and a silver/silver chloride electrode part supported by the base material.

12. The electrode according to claim 10, wherein the electrode is configured as a biological signal sensing electrode.

13. A conductive film comprising:
layered material particles comprising a plurality of layers, and a conductive polymer,
the plurality of layers comprising a layer body represented by:
MₘXₙ
wherein M is one or more metals selected from Group 3, 4, 5, 6, or 7 and includes Ti;
X is a carbon atom, a nitrogen atom, or a combination thereof,
n is not less than 1 and not more than 4; and
m is greater than n but not more than 5, and
a modification or termination T exists on a surface of the layer body, wherein T is at least one selected from the group consisting of a hydroxyl group, a fluorine atom, a chlorine atom, an oxygen atom, and a hydrogen atom, and
wherein a ratio of the layered material particles comprising the plurality of layers in the layered material particles included in the conductive film is 80 vol % or more.

14. The conductive film according to claim 13, wherein the conductive polymer is one or more selected from the group consisting of polythiophene, poly(3,4-ethylenedioxythiophene), polystyrene sulfonic acid, polyaniline, and polypyrrole.

15. The conductive film according to claim 13, wherein the layered material particle is a metal atom-containing layered material particle containing a metal atom.

16. The conductive film according to claim 15, wherein the metal atom is contained in the conductive film in an amount of 6.0×10⁻⁴ mol or more per 1 g of the metal atom-containing layered material particle.

17. The conductive film according to claim 13, wherein at least a part of an outermost surface of the layered material particles is covered with a conductive polymer.

18. A manufacturing method for a conductive film, the method comprising:
(a) preparing a precursor represented by:
MₘAXₙ
wherein M is one or more metals selected from Group 3, 4, 5, 6, or 7 and includes Ti;
X is a carbon atom, a nitrogen atom, or a combination thereof;
A is one or more elements selected from Group 12, 13, 14, 15, or 16;
n is not less than 1 and not more than 4; and
m is greater than n but not more than 5;
(b) removing at least a part of A atoms from the precursor by using an etching solution containing F⁻ to obtain an etching treatment product;
(c) water washing the etching treatment product without performing delamination to obtain layered material particles; and
(d) supplying a mixture of the layered material particles and a conductive polymer onto a base material to form a conductive film.

19. The manufacturing method according to claim 18, further comprising:
mixing a compound containing a metal atom with a precursor at the time of (b), or
mixing a compound containing a metal atom with the etching treatment product after (b) but before (c).

20. The manufacturing method according to claim 18, wherein the metal atom is one or more of lithium, sodium, and potassium.
